(19) United States
(12) Patent Application Publication
(10) Pub. No.: US 2026/0165743 A1
(43) Pub. Date: Jun. 18, 2026

(54) DISTRACTION LOSS MAGNET ON-OFF MECHANISM

(71) Applicant: NuVasive Specialized Orthopedics, INC., San Diego, CA (US)

(72) Inventor: Jorge Lopez Camacho, Oxnard, CA (US)

(21) Appl. No.: 19/533,978

(22) Filed: Feb. 9, 2026

Related U.S. Application Data
(63) Continuation of application No. 17/806,552, filed on Jun. 13, 2022, now Pat. No. 12,551,240.

(51) Int. Cl. A61B 17/68; A61B 5/151; A61B 17/00; A61B 17/17; A61B 17/52; A61B 17/56; A61B 17/66; A61B 17/70; A61B 17/72; A61B 34/00

(52) U.S. Cl. CPC A61B 17/68; A61B 34/73; A61B 5/15123; A61B 2017/00411; A61B 2017/00876; A61B 17/1707; A61B 17/52; A61B 2017/564; A61B 17/66; A61B 2017/681; A61B 17/7016; A61B 17/72; A61B 17/7216; A61B 17/7225; A61B 2034/731; A61B 2034/732; A61B 2034/733

(57) ABSTRACT

Disclosed herein are lock mechanisms configured for locking and unlocking rotation of a driver and a driven gear system in implantable distraction and compression systems, and implantable medical devices and implantable distraction and compression systems including such lock mechanisms. The lock mechanisms include a keeper that is configured to move from a locked position to an unlocked position in response to a rotation of the driver, wherein, in the locked position, the keeper is configured to resist rotation of the driven features and the driver under a load on the driven features, and in the unlocked position, permits rotation of the drive gear and the driver.

### CROSS-REFERENCE TO RELATED APPLICATIONS

This application is a continuation of U.S. patent application Ser. No. 17/806,552 filed on Jun. 13, 2022, which is incorporated in its entirety herein.

### FIELD OF THE INVENTION

The invention relates generally to implantable medical devices, and more particularly, to implantable distraction and compression devices, and rotation lock mechanisms for use therein, to prevent distraction or compression loss under load.

### BACKGROUND

Distraction osteogenesis is a technique which has been used to grow new bone in patients with a variety of defects. For example, limb lengthening is a technique in which the length of a bone, for example a femur or tibia, may be increased. By creating a corticotomy, or osteotomy in the bone, which is a cut through the bone, the two resulting sections of bone may be moved apart at a particular rate, for example one (1.0) mm per day, allowing new bone to regenerate between the two sections as they move apart. This technique of limb lengthening may be used in cases where one limb is longer than the other, such as a prior bone break that did not heal correctly, or in a patient whose growth plate was diseased or damaged prior to maturity. In some patients, stature lengthening is desired, and is achieved by lengthening both femurs and/or both tibias to increase the height of the patient.

In other clinical applications, treatment of an orthopedic condition may include compressing, retracting, or pulling portions of bone together. For example, in certain compressive applications, it may be desirable to hold or pull two fractured sections of a bone together.

Regardless of whether compression or distraction is performed, implantable compression and distraction devices may be exposed to significant loads placed on the drive features. These loads may undesirably cause back-driving and loss of previously achieved distraction or compression. It is therefore desirable to provide improved implants, systems, and devices which are capable of performing distraction and retraction/compression procedures while avoiding losses due to back-driving of the device under load. Desirably, the improved implants, systems, and devices are resistant to such losses while allowing the device to operate in both forward and reverse, i.e., to perform both distraction and compression, and to do so without compromising the efficiency of the device in the generation of distraction and compression forces.

### SUMMARY

A first aspect of the disclosure provides an implantable medical device comprising: a driver; a driven gear system; and a keeper, in which the keeper comprises: a body; a keyed opening in the body; an opening in the body configured to receive a center pin of the drive gear of the driven gear system; and a lock tooth disposed on a first end of the body. According to this aspect, the keyed opening may be configured to receive a complementarily keyed portion of a drive gear of the driven gear system, wherein the keeper and the drive gear are rotationally fixed to one another. The opening in the body may be configured to receive a center pin of the drive gear of the driven gear system, and to permit the body to translate axially relative to the center pin. The lock tooth may be configured to releasably mesh with teeth on a ring gear of the driven gear system. The keeper may be configured to move from a locked position to an unlocked position in response to a rotation of the driver. In the locked position, the keeper is configured to prevent rotation of the drive gear and the driver under a load on the driven gear system, and in the unlocked position, to permit rotation of the drive gear and the driver.

In certain embodiments, the keeper is further configured to rotate to move from the locked position to the unlocked position; to translate axially relative to the center pin in a direction opposite the first end in response to the rotating of the keeper; and to disengage the lock tooth from the ring gear of the driven gear system in response to the translating, thereby moving the keeper to the unlocked position.

In certain embodiments, the keeper further comprises a first face configured to engage the driver, and a second face configured to engage the driven gear system. The opening extends from the first face through the body to the second face, and the keyed opening extends from the second face through a partial thickness of the keeper. In certain embodiments, the opening and the keyed opening are fluidly coupled, wherein the keyed opening is bounded by drive surfaces configured to drive rotation of the keyed portion of the drive gear, and the opening is bounded by shaft relief surfaces configured to permit translation of the center pin. The keeper may further comprise a stepped surface between the drive surfaces and the shaft relief surfaces.

In certain embodiments, the keeper further comprises a first detent disposed on the first face. The first detent is disposed between the opening and the first end of the body, and comprises a first ramp surface. The keeper further comprises a second detent disposed on the first face at a second end thereof, opposite the first end. The second detent comprises a second ramp surface. Each of the first detent and the second detent may be configured to receive a drive pin disposed on an end of the driver.

In certain embodiments, the keeper further comprises a hole disposed in the second end of the body and open to the keyed opening, the hole being configured to receive a biaser. In the locked position, the lock tooth is configured to engage the ring gear, and to maintain such engagement under biasing force from the biaser in an expanded condition. In the unlocked position, the lock tooth is configured to disengage from the ring gear in response to a compression of the biaser upon rotation of the driver and the keeper.

In certain embodiments, the first ramp and the second ramp are each substantially v-shaped, and a zenith of the first ramp, a zenith of the second ramp, and the lock tooth are laterally aligned, such that the drive pins disposed in each of the first detent and the second detent are disposed in the zeniths of the first ramp and the second ramp when the keeper is in the locked position. The drive pins disposed in each of the first detent and the second detent may then move up the respective first ramp and second ramp when the keeper moves into the unlocked position.

In certain embodiments, the first detent and the second detent are further configured to permit rotation of the driver relative to the keeper to an extent limited by a length of the first ramp and the second ramp.

In certain embodiments, the first detent and the second detent each extend through a partial thickness of the keeper.

A second aspect of the disclosure provides a lock mechanism configured for locking and unlocking rotation of a driver and a driven gear system in an implantable distraction and compression system. The lock mechanism comprises: a driver of rotational motion, comprising a first drive pin and a second drive pin each extending axially from a first end of the driver; a drive gear configured to be driven by the driver, the drive gear comprising a keyed portion, and a keeper disposed over the drive gear and configured to move between a locked position and an unlocked position. The keeper comprises a keyed opening configured to rotatably engage the keyed portion of the keyed drive shaft, a lock tooth disposed on a first end of the keeper, the lock tooth being configured to releasably engage a ring gear of the driven gear system, a first detent and a second detent configured to engage the first drive pin and the second drive pin, and a biaser configured to bias the keeper across a longitudinal axis of the driver and the drive gear. In the locked position, the keeper resists rotation of the drive gear and the driver under a load on the driven gear system. In the unlocked position, the keeper permits rotation of the drive gear and the driver.

In certain embodiments, in the locked position, the biaser is configured to bias the keeper and the lock tooth into meshed engagement with the ring gear, and in the unlocked position, the biaser is configured to be compressed by the keeper upon rotation of the driver, thereby releasing engagement between the lock tooth and the ring gear and permitting the driver and the drive shaft to rotate.

In certain embodiments, the driver further comprises a central recess open to the first end of the driver, and disposed between the first drive pin and the second drive pin. The drive gear further comprises a central pin and a gear, each coupled to the keyed portion of the drive gear at opposing ends thereof. The central pin may be disposed at least partly within the central recess of the driver.

In certain embodiments, the lock mechanism further comprises a hole extending from the keyed opening through the second end of the keeper, and into the keyed portion, wherein the second end is opposite the first end, and wherein the biaser is disposed within the hole.

In certain embodiments, the lock mechanism further comprises a radial bearing configured to maintain a coaxial relationship between two or more of the driver, the keeper, and the keyed drive gear.

In certain embodiments, each of the first and the second detents comprise a ramp surface along which the respective drive pin is configured to travel.

In certain embodiments, the driver comprises a cylindrical permanent magnet configured to be rotated by the application of a magnetic field, and a magnet housing disposed about the cylindrical permanent magnet.

In certain embodiments, the gear is a sun gear, the driven gear system is a planetary gear system, and the sun gear is configured to engage the ring gear via a plurality of planetary gears.

In certain embodiments, the keyed portion has a square cross-sectional shape, and the keyed opening has a corresponding and complementary square cross-sectional shape.

A third aspect of the disclosure provides an implantable distraction and compression system comprising: a housing configured to be attached to a first bone portion, the housing having a driver, a driven gear system, and a lead screw positioned therein, wherein the lead screw is coupled to the driver via the driven gear system such that rotation of the driver causes rotation of the lead screw; a rod configured to be attached to a second bone portion and configured to interact with the lead screw such that, upon rotation of the lead screw, the rod distracts or contracts relative to the housing; and a lock mechanism configured to lock and unlock rotation of the driver and the driven gear system. The lock mechanism comprises a drive gear configured to be driven by the driver, and to input torque into the driven gear system, the drive gear comprising a keyed portion, and a keeper engaged with the driver and with the keyed portion to move between a locked position and an unlocked position in response to rotation of the driver. In the locked position, the keeper resists rotation of the drive gear and the driver under a load on the lead screw, and in the unlocked position, the keeper permits rotation of the drive gear and the driver.

These and other aspects, advantages and salient features of the invention will become apparent from the following detailed description, which, when taken in conjunction with the annexed drawings, where like parts are designated by like reference characters throughout the drawings, disclose embodiments of the invention.

### BRIEF DESCRIPTION OF THE DRAWINGS

FIG. 1 shows a top view of an exemplary distraction and compression device in accordance with an embodiment of the invention.

FIG. 2 shows a cross-sectional top view of a distraction and compression device in accordance with an embodiment of the invention.

FIG. 3 shows a perspective view of a lead screw in accordance with an embodiment of the invention.

FIG. 4 shows a cross-sectional view of a portion of a distraction and compression device in accordance with an embodiment of the invention.

FIG. 5 shows an enlarged perspective view of a retainer clip as deployed in the embodiments of FIGS. 2 and 4, among others, according to embodiments of the invention.

FIGS. 6-7 show enlarged perspective views of an anti-jam ring and an anti-jam retainer, respectively, as deployed in the embodiments of FIGS. 2 and 4, among others, according to embodiments of the invention.

FIGS. 8A-8D show enlarged perspective views of an anti-jam spring, as deployed in at least the embodiment of FIG. 9, according to embodiments of the invention.

FIG. 9 shows a cross-sectional view of a portion of an exemplary distraction and compression device including a lock mechanism in accordance with embodiments of the invention.

FIG. 10 shows an exploded perspective view of a gear assembly, a magnet assembly, and a magnet lock mechanism in accordance with an embodiment of the invention.

FIG. 11 shows an enlarged perspective view of the magnet housing of FIG. 10, in accordance with an embodiment of the invention.

FIGS. 12-13 show perspective views of a ring gear in accordance with an embodiment of the invention.

FIG. 14 shows a perspective view of a portion of the housing of an exemplary distraction and compression device in accordance with an embodiment of the invention.

FIGS. 15-17 show perspective, front, and back views of a keeper as deployed in the lock mechanism of at least FIG. 10, in accordance with embodiments of the invention.

FIGS. 18-19 show perspective views of a keyed drive gear as deployed in the lock mechanism of at least FIG. 10, in accordance with embodiments of the invention.

FIG. 20 shows a cross sectional view of a portion of an exemplary distraction and compression device including a lock mechanism in accordance with an embodiment of the invention.

FIGS. 21 and 22 show cross-sectional views taken along line A-A of FIG. 20, in which FIG. 21 depicts the locked position and FIG. 22 depicts the unlocked position, in accordance with embodiments of the invention.

FIG. 23 shows the internal components of an external adjustment device for non-invasively adjusting a distraction and compression device according to embodiments of the invention.

FIGS. 24-25 show external adjustment devices in configurations for adjusting a distraction and compression device implanted within a femur, and within a tibia, respectively, in accordance with embodiments of the invention.

FIG. 26 shows a cross sectional view of a portion of an exemplary distraction and compression device including a lock mechanism in accordance with an embodiment of the invention.

FIG. 27 shows an exploded perspective view of a magnet assembly and a magnet lock mechanism in accordance with an embodiment of the invention.

FIG. 28 shows an enlarged perspective view of the magnet housing of FIG. 27, in accordance with an embodiment of the invention.

FIGS. 29-31 show perspective, front, and back views of a keeper as deployed in the lock mechanism of at least FIG. 27, in accordance with embodiments of the invention.

FIGS. 32-33 show perspective views of a keyed drive output as deployed in the lock mechanism of at least FIG. 27, in accordance with embodiments of the invention.

FIG. 34 shows a cross sectional view of a portion of an exemplary distraction and compression device including a lock mechanism in accordance with an embodiment of the invention.

FIGS. 35 and 36 show cross-sectional views taken along line A-A of FIG. 34, in which FIG. 35 depicts the locked position and FIG. 36 depicts the unlocked position, in accordance with embodiments of the invention.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

As indicated above, aspects of the invention provide various embodiments of distraction and compression implants, including intramedullary, extramedullary, and spinal distraction implants, as well as methods and devices for preventing distraction or compression loss in such devices. Such implants, methods, and devices may provide improvements over prior implants, methods, and devices. For example, the presently disclosed implants, methods, and devices may be relevant to performing distraction and retraction/compression procedures while avoiding losses due to back-driving of the device under load, while avoiding, limiting, or preventing reductions in efficiency in the generation of distraction forces.

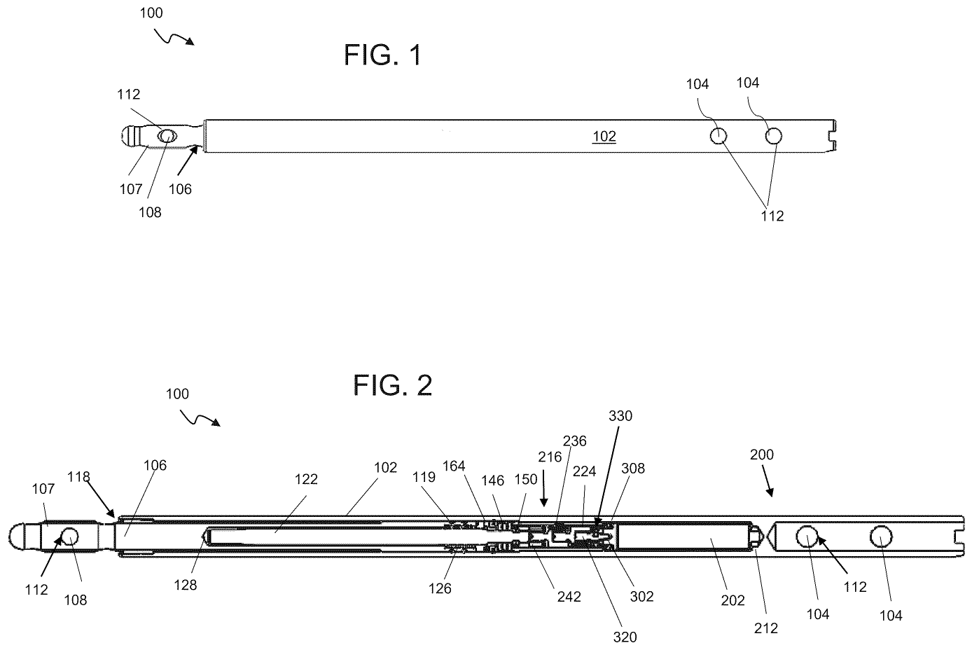

With reference to FIGS. 1-2, the present disclosure describes various embodiments of a distraction and compression device 100. As noted, the device 100 may be, for example, an extramedullary limb lengthening device, an intramedullary limb lengthening device, or a spinal distraction device. FIGS. 1 and 2 show a top view and a cross-sectional view, respectively, of an exemplary distraction and compression device 100 according to embodiments of the disclosure. As shown, the device 100 includes a housing 102 having at least one fixation aperture 104 and a distraction and compression rod 106 having at least one fixation aperture 108. Specifically, the rod 106 may terminate at a plate 107 having fixation apertures 108 therein. In certain embodiments, the plate 107 may be curved or angled in any of a number of different configurations depending on the location on the bone and the particular bone to which the device 100 is to be affixed, such that the plate 107 may be contoured to conform to a shape of the bone. Further, fixation apertures 104 may be positioned about a plate or bulge portion of the housing 102. At least one fixation aperture 104, 108 may particularly be a locking screw hole having internal threads 112 for engaging a thread disposed on a head of a fixation screw in a manner that will be readily understood by one of skill in the art. In some embodiments, all fixation apertures 104, 108 include internal threads 112.

The housing 102 is configured to be fixed to a bone at a first location and the rod 106 is configured to be fixed to the bone at a second location. In the context of limb lengthening, for example, in order to grow or lengthen bone, the bone may either have a pre-existing separation or may be purposely cut or broken (e.g., via an osteotomy) to create this separation, dividing the bone into a first section and a second section. The cut may be done prior to implanting and securing the device 100 or may be done after the device 100 is implanted, for example by use of a flexible Gigli saw. The rod 106 is configured to contract (e.g., for compression) and/or distract (e.g., for limb lengthening) relative to the housing 102. The device 100 is configured to allow controlled, precise translation of the rod 106 relative to the housing 102 by non-invasive remote control, and thus controlled, precise translation of the bone segment that is secured to the rod 106 relative to the bone segment that is secured to the housing 102.

Over the treatment period, for example for limb lengthening, the bone may be regularly distracted, creating a new separation, within which osteogenesis can occur. The term “regularly distracted” is meant to indicate that distraction occurs on a regular or periodic basis which may be on the order of, e.g., every day or every few days. An exemplary distraction rate may be one millimeter per day, although other distraction rates may be employed. That is to say, a typical distraction regimen may include a daily increase in the length of the device 100 by about one millimeter. This may be done, for example, by four lengthening periods per day, each having 0.25 mm of lengthening. The device 100 as disclosed herein includes a drive system which may be, for example, magnet-driven. The drive system allows the rod 106 to be telescopically extended from the housing 102, thus forcing the first section and the second section of the bone apart from one another.

As shown in FIG. 2, at one end, the housing 102 has an opening 118 for receiving the rod 106. The end of housing 102 which includes the opening 118 may be considered the distal end of housing 102. One or more o-rings 119 may be positioned about the rod 106, between the rod 106 and the housing 102. In some embodiments, a portion of the outer surface of the rod 106 and/or a portion of an internal surface of the housing 102 may be recessed to accommodate the o-ring(s) 119. The o-ring(s) 119 may facilitate proper sealing between the housing 102 and the rod 106 so that bodily fluid does not enter the housing 102 when the device 100 is implanted. The housing 102 is sealably closed at the end opposite opening 118, which may be referred to as the proximal end, by the attachment of an end cap. The end cap may be attached to the housing 102 by means of welding, adhesive bonding or other joining techniques. Further, another o-ring(s) may be provided between the end cap and the housing 102 to provide a seal therebetween.

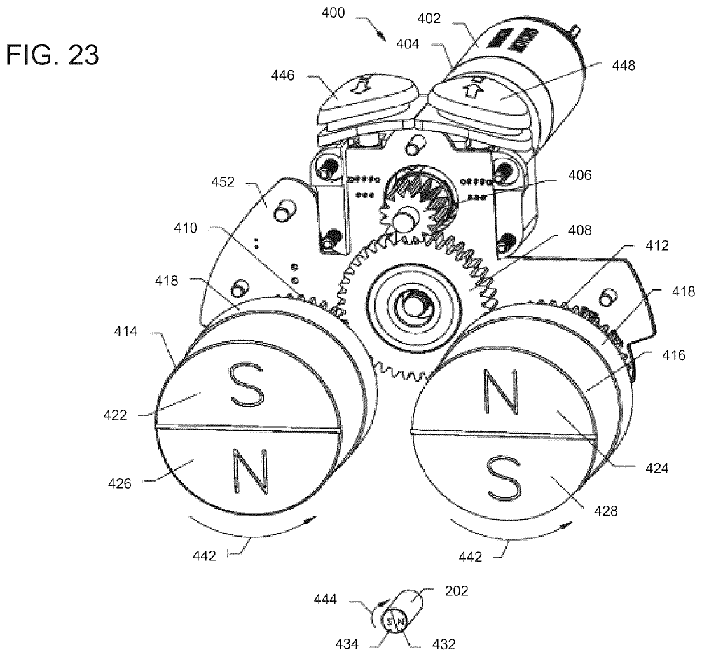

With reference to FIGS. 2-4, in use, the rod 106 is driven from the housing 102 by means of a lead screw 122 which turns inside a nut 126 that is secured to an inner surface of a cavity 128 in the rod 106 in which the lead screw 122 is disposed. The nut 126 is positioned between the lead screw 122 and the rod 106. The lead screw 122 is mechanically coupled in an indirect or direct manner, to a driver. The driver may be, for example, a cylindrical permanent magnet 202 contained within the housing 102, although in other embodiments, the driver may be, for example, a motor or other actuator as would be readily understood by one of skill in the art. The driver may be mechanically coupled to the lead screw 122 by a driven gear system as described further herein. In any event, rotation of the driver effectuates rotation of the lead screw 122, which translates into axial movement of the rod 106 relative to the housing 102. In embodiments in which the driver is a cylindrical permanent magnet 202, the rotation may be magnetically driven by an external adjustment device 400 (FIGS. 23-25),

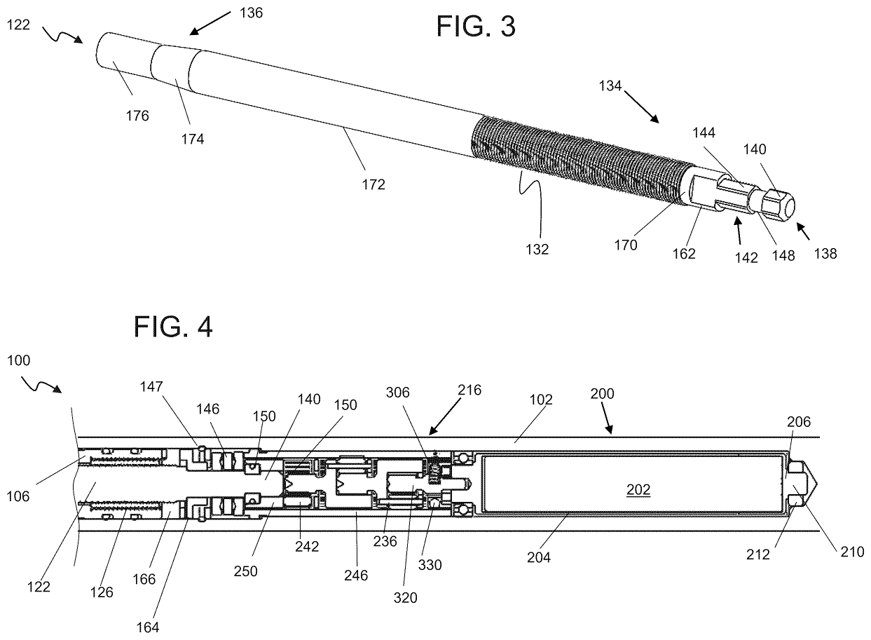

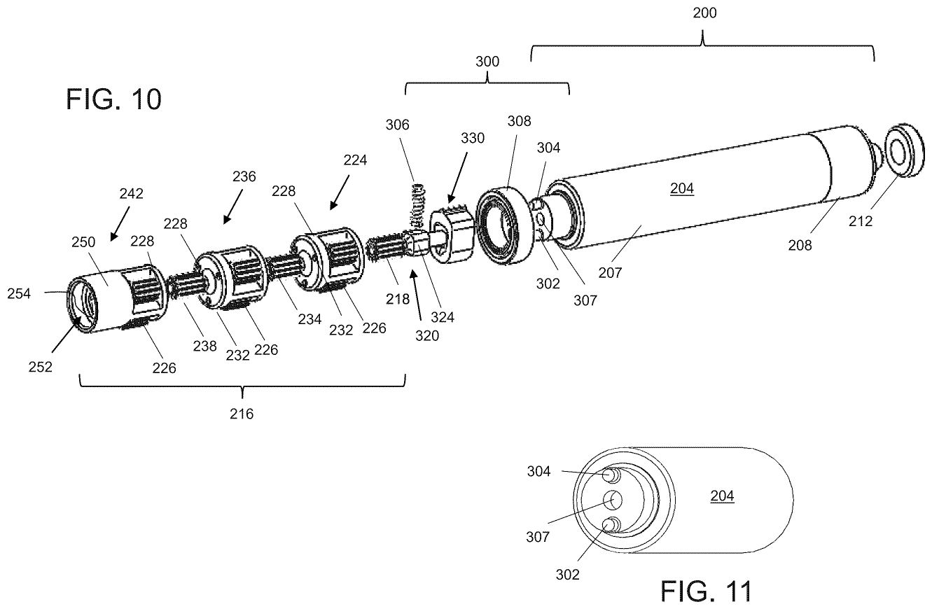

As shown in FIG. 3, the lead screw 122 includes a shaft 132 having a first end 134 and a second end 136. The first end 134 of the shaft 132 is configured to be coupled with the magnetic assembly 200 (see FIGS. 2 and 4). The first end 134 may include a first end portion 138 having an external keyed surface 140, which may for example be a hex shape, and a second portion 142 having an external keyed surface 144, which may for example be a hex shape. The first end portion 138 is configured to be engaged within an opening 252 (see FIG. 10) of the drive stage 250 (see FIGS. 4 and 10) of gear assembly 216 (FIGS. 2, 4, and 10) as will be described herein. The second portion 142 having the external keyed surface 144 is configured to be engaged with at least one thrust bearing 146 (see FIGS. 2, and 4, with two shown in FIG. 4) positioned adjacent to at least one retainer clip 150 (see for example FIGS. 2, 4, and 5, with two shown in FIG. 4). The thrust bearings 146 each consist of two separate races with ball bearings disposed between the two races. The thrust bearings 146 are configured to transmit high compressive forces during rotation of the lead screw 122 and axial movement of the rod 106 relative thereto. Further, as best seen in FIG. 4, the housing 102 may include a retainer 147 to serve as an abutment surface for the thrust bearings 146.

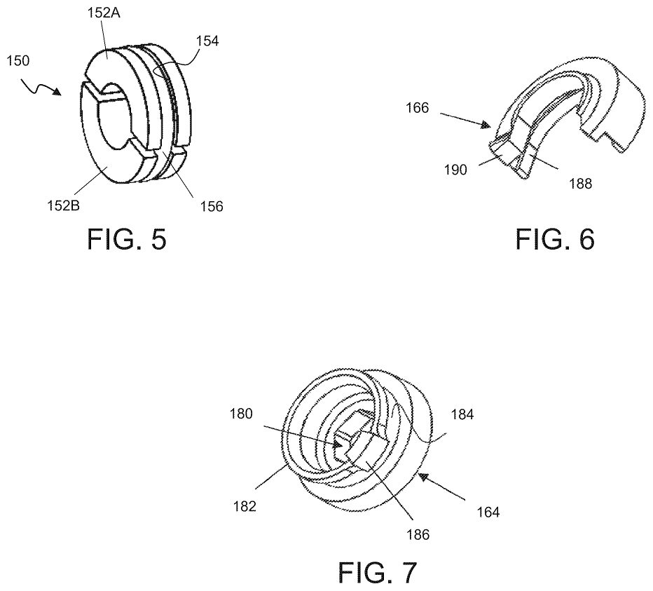

Returning to FIG. 3, between the two keyed surfaces 138, 142, the lead screw 122 is recessed to provide a smaller diameter potion 148. The smaller diameter portion 148 is sized and shaped to accommodate and/or engage with a retainer clip 150 (see FIGS. 4-5) such that the retainer clip 150 is disposed within the recess defined by the smaller diameter portion 148 between first and second keyed portions 138, 142.

FIG. 5 shows an enlarged view of the retainer clip 150. As shown, the retainer clip 150 includes two separate arcuate members 152A, 152B. Alternatively, the retainer clip 150 may include a single, unitary substantially cylindrical member structure. Arcuate or cylindrical members 152A, 152B may include a recess 154 disposed on a radially outer surface thereof, for seating of an o-ring 156 within the recess 154. The lead screw 122 is recessed on an opposing side of the second portion 142 from the smaller diameter portion 148 to provide another smaller diameter portion 162. Like portion 148, this portion 162 is also sized and shaped to accommodate and/or engage with a retainer clip 150 such that the retainer clip 150 is disposed within the recess defined by the smaller diameter portion 162. Further, the retainer clips 150 together with the retainer 147 of the housing 102 provide support for the thrust bearings 146 due to the retainer clips 150 being positioned about the lead screw 122 on opposing sides of the thrust bearings 146.

The lead screw 122 also includes a step 170 from the smaller diameter portion 162 to a larger diameter portion 172 (see FIG. 3). This larger diameter portion 172 is threaded along at least a portion of the axial extent thereof. In use, the threaded portion of larger diameter portion 172 is at least partially surrounded by the nut 126 (FIGS. 2 and 4) disposed within the cavity 128 within the rod 106 to facilitate axial movement of the rod 106 relative to the housing 102. The second end 136 of the lead screw 122 includes a ramp 174 that transitions from the larger diameter portion 172 to an intermediary diameter portion 176. The intermediary diameter portion 176 does not contain any threads thereon, so as the rod 106 engages this region of the lead screw 122, no additional movement of the rod 106 will be created. In certain embodiments, a stop (not shown) may be disposed on an end of intermediary portion 176 opposite ramp 174, the stop being configured to resist or prevent the rod 106 from disengaging from the lead screw 122 completely. The intermediary diameter portion 176 can be of a diameter smaller than the larger diameter portion 172 and larger than the small diameter portions 148, 162. However, it is also contemplated that the intermediary diameter portion 176 is of the same dimension as the smaller diameter portions 148, 162 or the larger diameter portions 172.

The device 100 may also include an anti-jam feature configured to prevent the rod 106 from jamming or stalling in a fully retracted state. Specifically, the anti-jam feature provides a spring force to overcome the friction force of the rod 106 in a scenario where the rod 106 jams or stalls.

In one embodiment, shown in FIGS. 4 and 6-7, the anti-jam feature may include a substantially circular anti-jam retainer 164 and an anti-jam ring 166 coupled thereto. The anti-jam ring 166 may be, for example, semi-circular or partially-circular. The anti-jam retainer 164 includes an opening 180 having a keyed shape, for example a hex shape, to complement the keyed shape 144 of the lead screw 122 at portion 142. This allows the anti-jam retainer 164 to be rotationally fixed relative to the lead screw 122, and therefore, as the lead screw 122 rotates, the anti-jam retainer 164 rotates with the lead screw 122. The anti-jam retainer 164 also includes a collar 182 for substantially surrounding the retainer clip 150 and maintaining a position of the retainer clip 150 relative to the lead screw 122. As shown, the internal surface of the collar 182 is substantially circular and does not have a keyed shape as in the opening 180. The collar 182 includes a groove 184 formed in an outer surface thereof. The collar 182 also includes a tab and/or projection 186 extending radially and axially therefrom. The anti-jam ring 166 is may include a flange 188 configured to be received within the groove 184 of the anti-jam retainer 164. The flange 188 has a width less than a width of the groove 184 such that slight axial movement of the flange 188 within the groove 184 is allowed, which therefore allows slight axial movement of the anti-jam ring 166 relative to the anti-jam retainer 164. The configuration of the flange 188 and the groove 184 maintains the coupling of the anti-jam ring 166 with the anti-jam retainer 164, while allowing the anti-jam ring 166 and the anti-jam retainer 164 to move rotationally and axially relative to each other. In addition, the anti-jam ring 166 includes a tab and/or a projection 190 extending axially and radially therefrom.

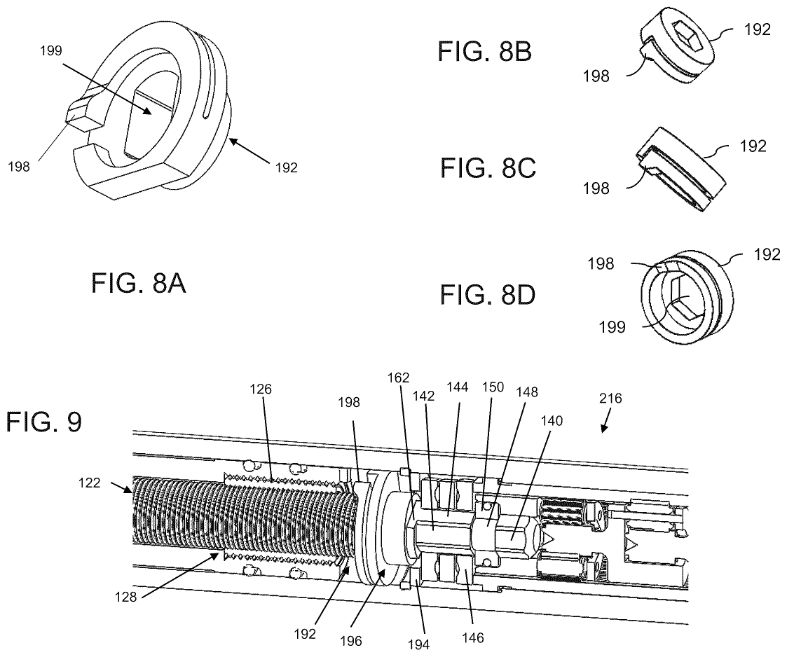

In another embodiment, shown in FIGS. 8A-8D and 9, the anti-jam feature described herein may be in the form of an anti-jam spring 192, which may be a one-piece helical spring as illustrated in FIGS. 8A-8D and 9, having a substantially round or oblong outer cross-sectional shape. In embodiments including an anti-jam spring 192, the distraction rod 106 may include a first tab protrusion 194 extending from a shoulder 196 adjacent an open end of the internal cavity 128 of the distraction shaft 106. The tab protrusion 194 is configured to cooperate with the anti-jam spring 192 as described herein.

The anti-jam spring 192 may include a second tab protrusion 198 configured to matingly engage the first tab protrusion 194 on the distraction shaft 106. Anti-jam spring 192 may further include a keyed opening 199 (see FIG. 8D) which may be, e.g., hexagonally shaped. The shape of keyed opening 199 is configured to complement a cross sectional shape of lead screw 122 at smaller diameter portion 162 (FIG. 9). In this manner, the anti-jam spring 192 is configured to be rotationally fixed to the lead screw 122. This configuration enables the anti-jam spring 192 to stay on-center relative to the lead screw assembly without tilting or floating, and remain constrained in a lead screw shoulder position. In use, when the anti-jam spring 192 is rotated by the lead screw 122, the tab 198 meets and engages the distraction rod tab 194. The resistance provided by the distraction rod tab 194 causes the anti-jam spring 192 to open into the inner diameter of housing 102, and provide the spring force to overcome surface friction as described above. The anti-jam spring 192 is further configured to maintain engagement and face contact with the shoulder 196 of the distraction rod 106 during full retraction of the distraction rod 106.

Regardless of the specific embodiment, the anti-jam feature serves to prevent the rod 106 from jamming or stalling in a fully retracted state. Specifically, in a jammed state, the external adjustment device 400 causes the lead screw 122 (via the magnet 202, shown in FIG. 4) to rotate but the rod 106 may not move axially due to being jammed. For example, a rod 106 may become jammed in a fully retracted state due to frictional forces in the retracted state. Therefore, a torque greater than a torque provided by the external adjustment device 400 (see FIGS. 23-25) may be needed to jumpstart or overcome the frictional forces in a jammed state. As a result, the anti-jam feature provides a built-in mechanism within the device 100 to provide an additional force above and beyond that which is provided by the external adjustment device 400, thereby providing such a jumpstart force.

Referring back to FIG. 4, a rotatable magnetic assembly 200 is located within the housing 102. The magnetic assembly 200 includes a cylindrical, radially-poled permanent magnet 202 contained within a magnet housing 204 having an end cap 206. The permanent magnet 202 may include rare earth magnet materials, such as Neodymium-Iron-Boron. The permanent magnet 202 may further have a protective Phenolic coating thereon, and may be held statically within the magnet housing 204 and end cap 206 by epoxy or other adhesive. The magnet housing 204, end cap 206 and epoxy form a seal to further protect the permanent magnet 202. The magnet housing 204 may also be welded to the end cap 206 to create a hermetic seal. To aid in manufacturing and assembly, the magnet housing 204 may include separate magnet cups 207, 208 (see FIG. 7) for housing the permanent magnet 202 therein. The end cap 206 includes a cylindrical extension or axle 210 which fits within the inner diameter of a radial bearing 212, allowing for low friction rotation.

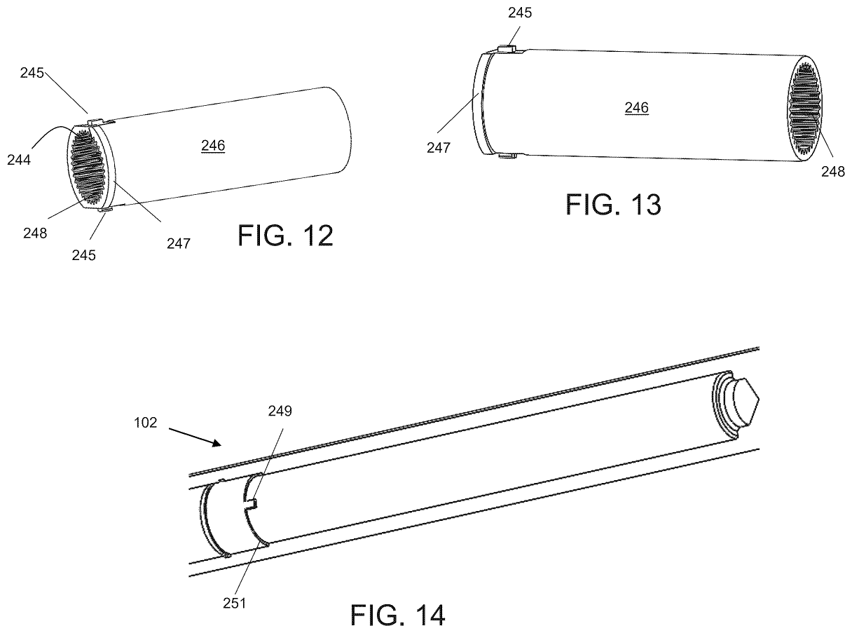

As shown in at least FIGS. 2, 4, 9, and 10, the magnetic assembly 200 may be coupled to a gear assembly 216, which in turn is configured to couple the lead screw 122 to the magnet assembly 200. Referring to FIG. 10, the magnetic assembly 200 may terminate with a first sun gear 218. The first sun gear 218 turns in a 1:1 fashion in response to rotation of the magnetic assembly 200, upon application of a moving magnetic field applied to the patient from an external location. The first sun gear 218 is configured to insert within an opening of a first gear stage 224 having three planetary gears 226 which are rotatably held in a frame 228 by axles 232. A second sun gear 234, which is the output of the first gear stage 224, turns with frame 228. The identical components exist in second gear stage 236, which outputs to a third sun gear 238, and third gear stage 242, which terminates in a drive stage 250. The drive stage 250 is positioned about the gear assembly 216 furthest from the magnet assembly 200. Along the length that the gear stages 224, 236, 242 extend, the inner wall 244 of a ring gear 246 (as seen in FIGS. 12-13) has internal teeth 248 along which the externally extending teeth of the planetary gears 226 engage as they turn. Each gear stage illustrated has a 4:1 gear ratio, so the drive stage 250 turns once for every 64 turns of the magnetic assembly 200.

The frame 228 of the third gear stage 242 includes the drive stage 250. The drive stage 250 includes an opening 252 having a keyed internal surface 254. The keyed internal surface 254 is configured to matingly engage with a keyed external surface 140 of the end 134 of the lead screw 122 (see FIG. 3). The engagement of the keyed surfaces 254, 140 prevent rotation of lead screw 122 and the drive stage 250 relative to each other. The keyed surfaces 254, 140 may be, for example, female and male hex shapes, respectively. However, other shapes that prevent rotation of the lead screw 122 relative to the drive stage 250 are also contemplated by the disclosure. To further maintain the lead screw 122 within the drive stage 250, a first retainer clip 150 may be provided within the opening 252 of the drive stage 250 and at least partially surrounding the lead screw 122 proximal to the keyed external surface 140. Specifically, the retainer clip 150 may be positioned about the smaller diameter portion 148 (see FIG. 3) within the opening 252.

The torque applied on the magnetic assembly 200 by the action of the rotating magnetic field on the cylindrical permanent magnet 202, is therefore augmented on the order of 64 times in terms of the turning torque of the lead screw 122. This allows the rod 106 to be able to move with high precision. Because of the 64:1 gear ratio, the device 100 is able to axially displace the bone segment coupled to the rod 106 against severe resisting forces, for example those created by soft tissue.

As shown in FIGS. 2, 4, and 9, one or more thrust bearings 146 serve to protect the magnet assembly 200 and the gear assembly 216 from any significant compressive or tensile stresses. When there is a compressive force on the device, for example, when distracting a bone, and thus resisting the tensile strength of the soft tissues, the thrust bearing(s) 146 abuts against retainer clip(s) 150 and/or retainer 147. In other embodiments, the device 100 is used for pulling bones together. For example, in certain compressive applications, it is the goal to hold two fractured sections of a bone together. In these compressive applications, the device 100 may be under a tensile force and the thrust bearing(s) 146 would abut against the retainer clip(s) 150 or retainer 147. In both situations, the thrust bearings 146 and the retainer 147 absorb the large stresses, rather than the magnet assembly 200 or gear assembly 216 of the drive system.

With further reference to FIGS. 12-13, as shown, the ring gear 246 includes an inner wall 244 having internal teeth 248 as well as at least one raised portion 245 and at least one tab and/or projection 247. In particular, the ring gear 246 may include two tabs or projections 247. The raised portion 245 is positioned on an external surface of the ring gear 246 such that the raised portion 245 extends radially from the external surface of the ring gear 246. In some embodiments, the ring gear 246 can include two raised portions 245 positioned on opposing sides of the ring gear 245. The raised portion 245 engages and/or mates with a complementary cutout or depression 249 (FIG. 14) formed within the housing 102 on an internal surface thereof, at a location where the housing 102 surrounds the ring gear 246. Where two opposing raised portions 245 are included on the ring gear 246, the housing 102 can include two opposing complementary cutouts or depressions 249. Any number of raised portions 245 and complementary cutouts 249 can be included without departing from aspects of the disclosure. Further, it is contemplated that an alternative configuration is equally applicable, as the housing 102 can include one or more raised portions that engage with and/or mate with complementary cutouts or depressions on the ring gear 246.

The tab 247 of the ring gear 246 can be positioned about an end of the ring gear 246 and extend radially therefrom. The tab 247 may engage and/or mate with a complementary groove 251 formed within the housing 102 on an internal surface thereof, where the housing 102 surrounds the ring gear 246 (see FIG. 14). It is to be understood that any number of tabs and/or projections 247 and complementary grooves 251 can be included without departing from aspects of the disclosure. For example, the ring gear 246 can include two tabs 247 on opposing sides of the ring gear 246. As shown, in some embodiments, each tab 247 may be positioned between the raised portions 245 such that tabs and raised portions 245 alternate about the ring gear 246. Further, it is contemplated that an alternative configuration is equally applicable, as the housing 102 can include tabs and/or projections that engage with and/or mate with complementary grooves on the ring gear 246. These complementary features on the ring gear 246 and housing 102 prevent the ring gear 246 from moving axially relative to the housing 102.

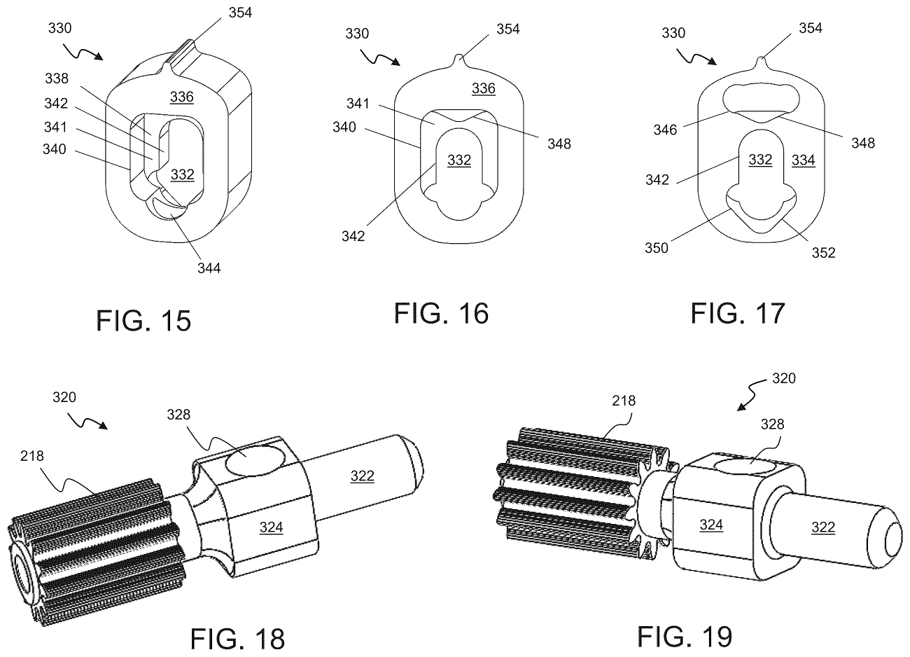

Referring back to FIGS. 10-11, and referring also to FIGS. 15-22, the device 100 may include a lock mechanism 300, which may be configured to lock and unlock rotation of a driver and a driven gear system. The lock mechanism 300 may be configured to allow the driver to drive in either forward or reverse, i.e., to rotate in either direction depending on the desired distraction or compression application, in response to rotation of the driver. Thus, in the unlocked position, the lock mechanism 300 permits the driver and the driven gear system to rotate in either a clockwise direction or a counterclockwise direction as actuated by the driver. However, in the locked position, the lock resists, and in some embodiments prevents entirely, any rotation in the absence of rotation of the driver. As a result, the lock mechanism 300 resists, reduces, minimizes, or prevents back-driving and distraction loss caused by loads on the lead screw 122 when in the locked position.

In various embodiments, the driver may be any actuator of rotational motion. In the embodiment depicted in FIG. 10, the driver is a magnetic assembly 200 as described herein, including a cylindrical permanent magnet 202 that is configured to be rotated by the application of a magnetic field, and a magnet housing 204 disposed about the cylindrical permanent magnet 202. However, in other embodiments, the driver may be, e.g., a motor. Further, in various embodiments, the driven gear system may be a gear assembly 216 as described herein, and may particularly be a planetary gear system including a sun gear 218 that is configured to engage a ring gear 246 via a plurality of planetary gears 226 (see FIG. 10). However, in other embodiments, the driven gear assembly may be, for example, a cycloid gear assembly. In still other embodiments, the magnet assembly may engage the lead screw 122 direction, without a driven gear assembly interposed between the magnet assembly and the lead screw.

As noted, FIGS. 10-11 illustrate an embodiment in which the driver is in the form of a cylindrical permanent magnet 202 disposed within a magnet housing 204. The magnet housing 204 includes a first drive pin 302 and a second drive pin 304. As best shown in FIG. 11, each of the first and second drive pins 302, 304 extends axially from a first end of the magnet housing 204, in a direction parallel to a longitudinal axis of the cylindrical magnet 202. The first and second drive pins 302, 304 may be arranged on the end face of the magnet housing 204 such that they are disposed substantially opposite one another, spaced about 180 degrees apart from one another on the end face of the magnet housing 204. The magnet housing 204 may further include a central recess 307 that is open to the first end of the magnet housing 204, and is disposed between the first drive pin 302 and the second drive pin 304. The central recess 307 may be centered on, or concentric with the longitudinal, i.e. rotational axis of the cylindrical magnet 202 and the magnet housing 204.

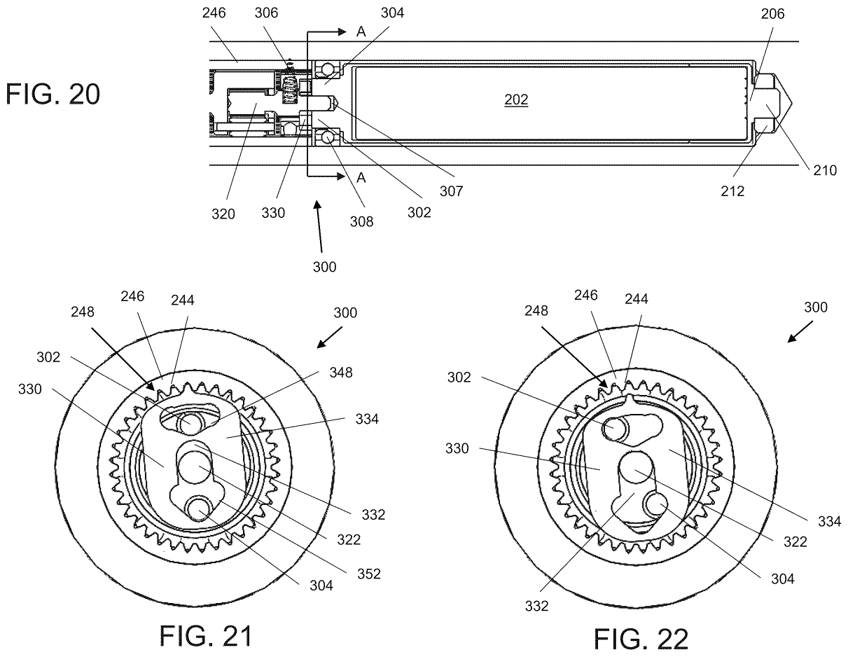

Referring back to FIG. 10, a keyed drive gear 320 is configured to be driven by the driver, e.g., by magnet housing 204, via the keeper 330, discussed further herein. The keyed drive gear 320, shown in detail in FIGS. 18-19, includes a central pin 322 coupled to a keyed portion 324, and a gear 218 coupled to the keyed portion 324 at an end opposite the end at which the keyed portion 324 is coupled to the central pin 322. Thus, the keyed portion 324 is disposed between the central pin 322 and the gear 218, each of which is coupled to the keyed portion 324 of the drive gear 320 at opposing ends thereof. The central pin 322, the keyed portion 324, and the gear 218 may be substantially coaxial with one another. As noted above, the gear 218 may be a sun gear in a planetary gear system. When assembled as shown in FIG. 20, the central pin 322 is configured to be disposed at least partly within the central recess 307 of the magnet housing 204. The keyed portion 324 may have any non-circular keyed cross-sectional geometry such as, for example, square, hexagon, rectangle, star, etc. In particular embodiments, the keyed portion 324 may have a square cross-sectional geometry.

A keeper 330 is disposed over at least a portion of the drive gear 320, and is interposed between the drive gear 320 and the driver. The keeper 330 is configured to move, e.g. by rotating, from a locked position to an unlocked position in response to rotation of the driver, e.g., magnet housing 204. In the locked position, the keeper 330 is configured to resist, reduce, minimize, or in certain embodiments prevent rotation of the drive gear 320 and the magnet housing 204 under a load on the driven gear system 216, and in the unlocked position, the keeper 330 is configured to permit rotation of the drive gear 320 and the driver, e.g., magnet housing 204.

As best seen in FIGS. 15-17, the keeper 330 includes a body having a first face 334 and a second face 336, where the first face 334 is configured to engage the driver, e.g., the magnet housing 204, and the second face 336 is configured to engage the driven gear system via the keyed drive gear 320.

A keyed opening 341 is disposed within the body and is open to the second face 336. The keyed opening 341 is configured to receive and rotatably engage the keyed portion 324 of the drive gear 320. The keyed opening 341 may extend from the second face 336 of the body of keeper 330 through a partial thickness of the keeper 330. The keyed opening 341 is shaped and dimensioned to provide a complementary fit with the keyed portion 324 of the drive gear 320, such that, for example, the keyed portion 324 fits in male/female engagement within the keyed opening 341 of the keeper 330. Where, for example, the keyed portion 324 has a square, hexagon, rectangle, star, or other shape, the keyed opening 341 may have a corresponding square, hexagon, rectangle, star, or other shape that is dimensioned to receive the complementary square, hexagon, rectangle, star, or other keyed shape with a close fit. As a result, the keeper 330 and the drive gear 320 are rotationally fixed to one another via the keyed opening 341. For example, in embodiments in which the keyed portion 324 is square shaped, the keyed opening 341 may also be square shaped.

The keeper 330 may also include an opening 332 in the body, which is configured to receive the center pin 322 of the drive gear 320. The opening may extend from the first face 334 of the keeper 330 through at least a partial thickness of the body. The opening 332 and the keyed opening 341 may be fluidly coupled with one another, such that the opening 332 and the keyed opening 341 may partially overlap with one another. Therefore, the opening 332 may effectively extend from the first face 334 through a full thickness of the body of the keeper 330 to the second face 336 in certain areas. The keyed opening 341 may be bounded by drive surfaces 340, which are configured to drive rotation of the keyed portion 324 of the drive gear 320, while the opening 332 may be bounded by shaft relief surfaces 342, which are configured to permit translation of the center pin 322. In particular, the opening 332 may include an oblong or slot-shaped portion defined by the shaft relief surfaces 342, as shown in FIGS. 17, 21, and 22. This oblong shape may permit the body of the keeper 330 to translate relative to the center pin 322 in operation, as illustrated in the change in relative position of center pin 322 and opening 332 from the locked position of FIG. 21 to the unlocked position of FIG. 22. The keeper 330 may further include a stepped surface 338, shown in FIG. 15, disposed between the drive surfaces 340 and the shaft relief surfaces 342.

The keeper 330 further includes at least one lock tooth 354 disposed on a first end of the keeper 330, and extending axially therefrom. The lock tooth 354 is configured to releasably mesh with or engage the internal teeth 248 on the inner wall 244 of a ring gear 248 of the driven gear system in use (see FIGS. 21-22).

The keeper 330 may further include a first detent 346 disposed on the first face 334 of the keeper 330, the first detent 346 being configured to engage the first drive pin 302. The first detent 346 may particularly be disposed between the opening 332 and the first end of the body (which includes the lock tooth 354). The keeper 330 may further include a second detent 350, also disposed on the first face 334 of the keeper 330, the second detent 350 being configured to engage the second drive pin 304. The second detent 350 may particularly be disposed at the second end of the keeper 330, opposite the first end, and in fluid connection with the opening 332. It is noted that the first detent 346 may alternatively receive the second drive pin 304, and the second detent 350 may receive the first drive pin 302, to the same result.

Each of the first detent 346 and the second detent 350 may include a respective ramp surface 348, 352, along which the respective drive pin 302, 304 is configured to travel. The first ramp 348 and second ramp 352 may each be configured to be curved or angled, having approximately or substantially a u-shaped or v-shaped ramp surface. Each ramp 348, 352 may include a zenith which is laterally aligned with one another, and are also laterally aligned with the lock tooth 354 on the keeper 330. Accordingly, the drive pins 302, 304 disposed in each of the first detent 346 and the second detent 350 are disposed in the zeniths of the first ramp 348 and the second ramp 352 when the keeper 330 is in the locked position. The drive pins 302, 304 disposed in each of the respective first and second detents 346, 350 are configured to move up the respective first ramp 348 and the second ramp 352 when the keeper 330 moves into the unlocked position. In this manner, the first detent 346 and the second detent 350 are configured to permit rotation of the driver, e.g., magnetic housing 204, relative to the keeper 330 to an extent limited by a length of the first ramp 348 and the second ramp 352. In certain embodiments, the first detent 346 and the second detent 350 each extend through a partial thickness of the keeper 330.

As illustrated in FIG. 15, the keeper 330 further includes a hole 344 extending from the keyed opening 341 through the second end of the keeper 330. The second end of the keeper 330 is the end opposite the first end, on which the lock tooth 354 is disposed. Keyed portion 324 of drive gear 320 also includes a hole 328 (see FIGS. 18-19) which, when assembled as in FIG. 20, is aligned with hole 344. Thus, the holes 344 and 328 in the keeper 330 and the keyed portion 324 of the drive gear 320 form a substantially continuous hole or channel. The lock mechanism 300 further includes a biaser 306 disposed within the aligned holes 344 and 328. The biaser 306 may be, for example, a spring. The biaser 306 may be configured to bias the keeper 330 across, or in a direction substantially perpendicular to a longitudinal axis of the magnet housing 204 and the drive gear 320. With reference to FIG. 10, the lock mechanism 300 may further include a radial bearing 308, which may be disposed about at least a portion of the magnet housing 204, the keeper 330, and the keyed drive gear 320, and may be configured to maintain a coaxial relationship between two or more of an end portion of the magnet housing 204, the keeper 330, and the keyed drive gear 320.

In operation, the lock mechanism 300 including the keeper 330, defaults to a locked position when the driver, e.g., magnet housing 204, is stationary (see FIG. 21). In the locked position, the biaser 306 is configured to bias the keeper 330 relative to the ring gear 246 and the drive gear 320, such that the lock tooth 354 on the keeper 330 is biased into meshed engagement with the teeth 248 on the inner surface 244 of the ring gear 246. In particular, the lock tooth 354 may be maintained in engagement between two teeth 248 of the ring gear 246 under the biasing force of biaser 306 in its expanded condition. This biasing force may be applied across or perpendicular to the longitudinal axis of the magnet housing 204 and the keyed drive gear 320. Due to the geometry of the detents, the first and second drive pins 302, 304 are disposed in the zenith positions of each of the respective ramps 348, 352 in the locked position. In this position, the keeper 330 resists rotation of the drive gear 320 and the magnet housing 204. This resistance to rotation is independent of any load on the driven gear system.

Upon, and in response to rotation of the driver, e.g., the magnet housing 204, the keeper 330 transitions from the locked position to the unlocked position. As the magnet housing 204 rotates, either in a clockwise or counterclockwise direction, the drive pins 302, 304 move out of the zenith position following the curve or slope of ramps 348, 352. In the embodiment shown in FIG. 22, the magnet housing 204 has rotated in a counter clockwise direction, although the keeper 330 would function analogously if the rotation were clockwise. This rotation causes the keeper 330 to translate axially relative to the center pin 322 in the direction of the second end, opposite the first end, urged by the interaction between the ramps 348, 352 and the drive pins 302, 304. Relative to the translation of the keeper 330 toward the unlocked position, the lock tooth 354 is disposed on a trailing end of the keeper 330. This translation of the keeper 330 compresses the biaser, and causes the lock tooth 354 to disengage from the ring gear 246. With the lock tooth 354 now disengaged from the teeth 248 of the ring gear 246, the keeper 330 moves to its unlocked position, in which the keyed drive gear 320, and therefore the magnet housing 204 and the driven gear assembly 216, are free to rotate relative to the ring gear 246.

Upon, and in response to a cessation of rotation of the magnet housing 204, the opposite process occurs. In the absence of rotation of the magnet housing 204, and under the biasing force of biaser 306, the drive pins 302, 304 move downward along the ramps 348, 352 to return to the zenith position depicted in FIG. 21, and the keeper 330 translates axially relative to the central pin 322. The lock tooth 354 is on the leading end of the keeper 330 with respect to this translation motion toward the locked position. Lock tooth 354 engages the teeth 348 of the ring gear 246, thereby preventing rotation of the keeper 330, and therefore the keyed drive gear 320, which is rotationally fixed to the keeper 330. When the keyed drive gear 320 is rotationally locked, so too are the sun gear 218, the balance of gear assembly 216, and the lead screw 122. In this manner, regardless or independently of the load placed on the lead screw 122 and the gear assembly 216, distraction or compression loss is resisted, and may in some embodiments be prevented.

FIGS. 23-25 illustrate an external adjustment device 400 configured for applying a moving magnetic field to allow for non-invasive adjustment of the device 100 by turning a permanent magnet 202 within the device 100, as described. FIG. 23 illustrates the internal components of the external adjustment device 400, and for clear reference, shows the permanent magnet 202 of the device 100 without the rest of the assembly. The internal working components of the external adjustment device 400 may, in certain embodiments, be similar to those described in U.S. Patent Application Publication No. 2012/0004494, which is incorporated by reference herein. A motor 402 with a gear box 404 outputs to a motor gear 406. The motor gear 406 engages and turns central (idler) gear 408, which has the appropriate number of teeth to turn first and second magnet gears 410, 412 at identical rotational speeds. First and second magnets 414, 416 turn in unison with the first and second magnet gears 410, 412, respectively. Each magnet 414, 416 is held within a respective magnet cup 418 (shown partially). An exemplary rotational speed may be 60 RPM or less. This speed range may be configured to limit the amount of current density induced in the body tissue and fluids, to meet international guidelines or standards. As seen in FIG. 23, the south pole 422 of the first magnet 414 is oriented the same as the north pole 424 of the second magnet 416, and likewise, the first magnet 414 has its north pole 426 oriented the same as the south pole 428 of the second magnet 416. As these two magnets 414, 416 turn synchronously together, they apply a complementary and additive moving magnetic field to the radially-poled, permanent magnet 202, having a north pole 432 and a south pole 434. Magnets having multiple north poles (for example, two) and multiple south poles (for example, two) are also contemplated in each of the devices. As the two magnets 414, 416 turn in a first rotational direction 442 (e.g., counter-clockwise), the magnetic coupling causes the permanent magnet 202 to turn in a second, opposite rotational direction 444 (e.g., clockwise). The rotational direction of the motor 402 and corresponding rotational direction of the magnets 414, 416 is controlled by buttons 446, 448. One or more circuit boards 452 contain control circuitry for both sensing rotation of the magnets 414, 416 and controlling the rotation of the magnets 414, 416.

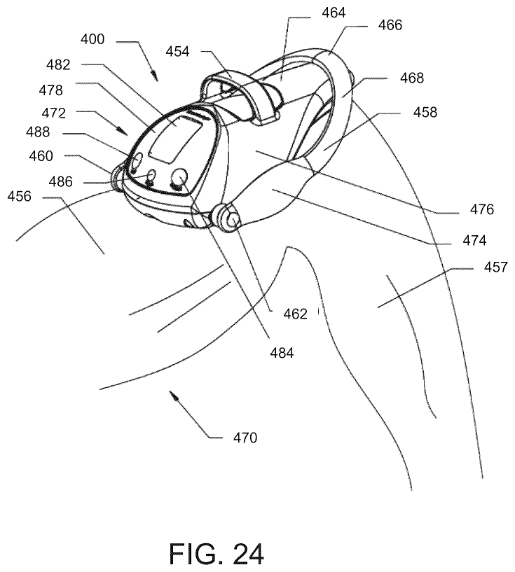

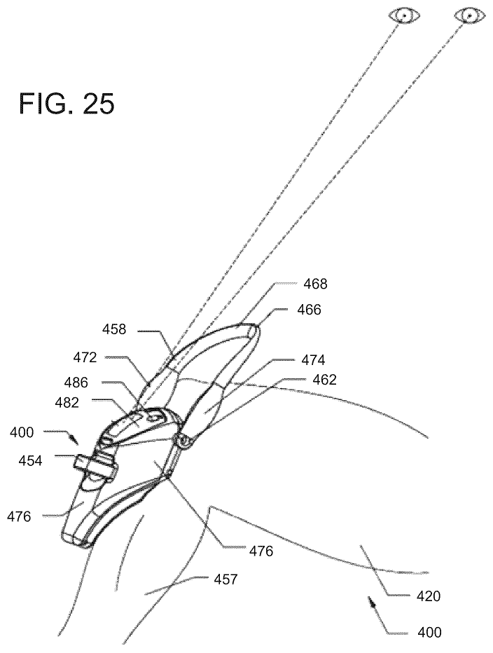

FIGS. 24 and 25 show the external adjustment device 400 for use with a device 100 placed in the femur (FIG. 24) or the tibia (FIG. 25). The external adjustment device 400 has a first handle 454 for carrying or for steadying the external adjustment device 400, for example, steadying it against an upper leg 456 (as in FIG. 24) or lower leg 457 as in (FIG. 25). An adjustable handle 458 is rotationally attached to the external adjustment device 400 at pivot points 460, 462. Pivot points 460, 462 have easily lockable/unlockable mechanisms, such as a spring-loaded brake, ratchet or tightening screw, so that a desired angulation of the adjustable handle 458 in relation to housing 464 can be adjusted and locked in orientation. Adjustable handle 458 is shown in two different positions in FIGS. 24 and 25. In FIG. 24, adjustable handle 458 is set so that apex 466 of loop 468 rests against housing 464. In this position, patient 470 is able to hold onto one or both of grips 472, 474 while the adjustment procedure (for example transporting bone between 0.10 mm to 1.50 mm) is taking place. It is contemplated that the procedure could also be a lengthening procedure for a bone lengthening device or a lengthening procedure for a lengthening plate which is attached external to the bone. Turning to FIG. 25, when the bone transport device 100 is implanted in a tibia, the adjustable handle 458 may be changed to a position in which the patient 470 can grip onto the apex 466 so that the magnet area 476 of the external adjustment device 400 is held over the portion of the device 100 containing the permanent magnet 202. In both cases, the patient 470 is able to clearly view control panel 478 including a display 482. In a different configuration from the two directional buttons 414, 416 in FIG. 24, the control panel 478 includes a start button 484, a stop button 486 and a mode button 488. Control circuitry contained on circuit boards 452 may be used by the surgeon to store important information related to the specific aspects of each particular patient. For example, in some patients an implant may be placed antegrade into the tibia. In other patients the implant may be placed either antegrade or retrograde about the femur. In each of these three cases, it may be desired to move the bone either from distal to proximal or from proximal to distal. By having the ability to store information of this sort that is specific to each particular patient within the external adjustment device 400, the external adjustment device 400 can be configured to direct the magnets 414, 416 to turn in the correct direction automatically, while the patient need only place the external adjustment device 400 at the desired position, and push the start button 484. The information of the maximum allowable bone transport length per day and maximum allowable bone transport length per session can also be input and stored by the surgeon for safety purposes. These may also be added via an SD card or USB device, or by wireless input. An additional feature is a camera at the portion of the external adjustment device 400 that is placed over the skin. For example, the camera may be located between first magnet 414 and second magnet 416. The skin directly over the implanted permanent magnet 202 may be marked with indelible ink. A live image from the camera is then displayed on the display 482 of the control panel 478, allowing the user to place the first and second magnets 414, 416 directly over the area marked on the skin. Crosshairs can be overlaid on the display 482 over the live image, allowing the user to align the mark on the skin between the crosshairs, and thus optimally place the external adjustment device 400.

Other external adjustment devices can be used to cause actuation of the distraction devices described herein. Such external adjustment devices include, for example, those described in U.S. Pat. No. 8,382,756 filed on Nov. 20, 2009, U.S. Pat. No. 9,248,043 filed Jun. 29, 2011, U.S. Pat. No. 9,078,711 filed on Jun. 6, 2012, U.S. Pat. No. 9,044,281 filed on Oct. 18, 2012, U.S. application Ser. No. 14/698,665 filed on Apr. 28, 2015, U.S. application Ser. No. 14/932,904 filed on Nov. 4, 2015, U.S. Ser. No. 16/004,099 filed on Dec. 12, 2016, and App. No. PCT/US2020/017338 filed on Feb. 7, 2020, all of which are incorporated herein by reference as if set forth in their entirety.

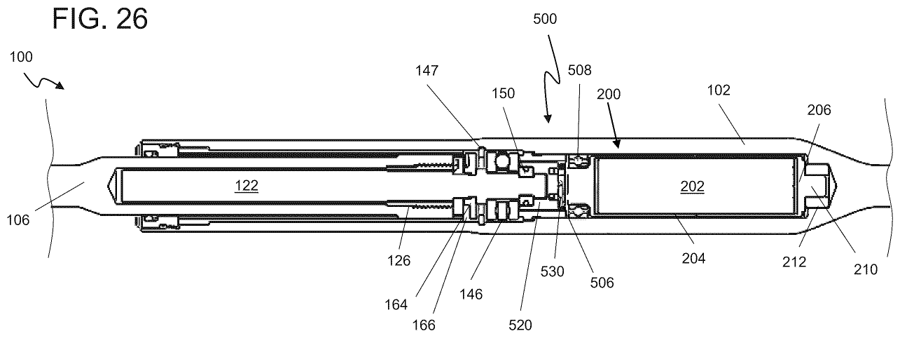

With reference to FIGS. 26-33, device 100 may include a lock mechanism 500, which may provide an alternative embodiment to the devices 100 including the lock mechanism 300 described previously. Like the first embodiment of a lock mechanism 300, the second embodiment of a lock mechanism 500 is configured to lock and unlock rotation of a driver and a lead screw 122. The lock mechanism 500 may be configured to allow the driver to drive in either forward or reverse, i.e., to rotate in either clockwise or counterclockwise direction depending on the desired distraction or compression application, in response to rotation of the driver. Thus, in the unlocked position, the lock mechanism 500 permits the driver and the lead screw to rotate in either a clockwise direction or a counterclockwise direction as actuated by the driver. However, in the locked position, the lock mechanism 500 resists, and in some embodiments prevents entirely, any rotation in the absence of rotation of the driver. As a result, the lock mechanism 500 resists, reduces, minimizes, or prevents distraction loss caused by loads on the lead screw 122 when in the locked position.

In various embodiments, the driver may be any actuator of rotational motion. For example, in the embodiment depicted in FIGS. 26-28 and 34, the driver is a magnetic assembly 200 as described herein, including a cylindrical permanent magnet 202 that is configured to be rotated by the application of a magnetic field, and a magnet housing 204 may be disposed about the cylindrical permanent magnet 202. The magnet housing 204 may be made up of separate magnet cups 207, 208 as described previously. In other embodiments, the driver may be, e.g., a motor.

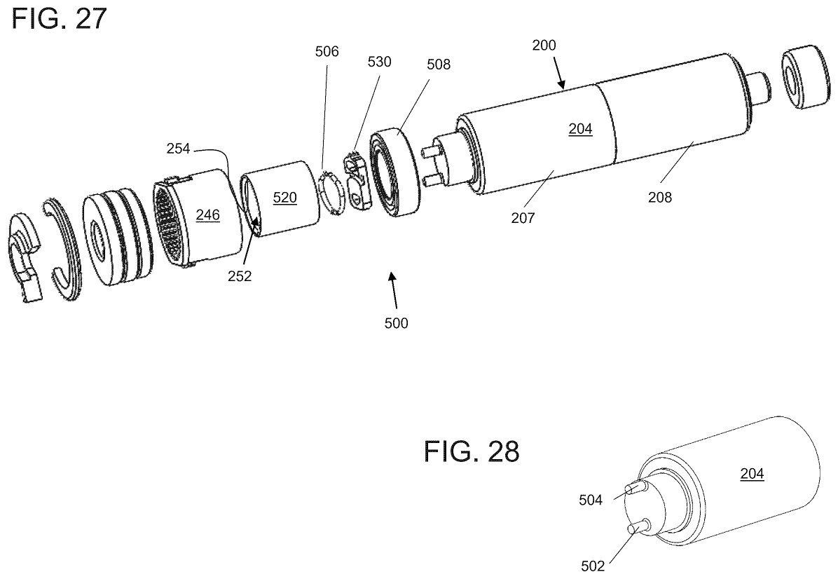

As noted, FIGS. 27-28 illustrate an embodiment in which the driver is in the form of a cylindrical permanent magnet 202 disposed within a magnet housing 204. The magnet housing 204 includes a first drive pin 502 and a second drive pin 504. As best shown in FIG. 28, each of the first and second drive pins 502, 504 extends axially from a first end of the magnet housing 204, in a direction parallel to the axis of rotation of the cylindrical permanent magnet 202. The first and second drive pins 502, 504 may be arranged such that they are disposed substantially opposite one another, spaced about 180 degrees apart from one another on the end face of the magnet housing 204.

A keyed drive stage 520, shown in detail in FIGS. 32-33 and in context in FIGS. 26, 27, and 34-36, is configured to be driven by the driver, e.g., by the magnet housing 204. The keyed drive stage 520 includes, on a torque input end, a first rotational slot 522 and a second rotational slot 524, each configured to receive one of the first and second drive pins 502, 504. As such, first and second rotational slots 522 and 524 may be arranged such that they are disposed substantially opposite one another, spaced about 180 degrees apart from one another on the input end of the keyed drive stage 520. Each of the first and second rotational slots 522 and 524 may have an arcuate shape, configured to allow a limited amount of rotation of the driver relative to the rotational slots 522, 524. The input end may further include a recess 527 shaped and dimensioned to receive a keeper 530 as described herein, and to allow the keeper 530 to translate across a rotational axis of the keyed drive stage 520 as further described herein. Still further, the recess 527 may include vertical guide surfaces 529 configured to define or guide the keeper 530 on the translational path of the keeper 530. The recess 527 may further be configured to allow the keeper 530 to translate beyond or across the outer circumference of the end of the keyed drive stage 520, to allow the keeper 530 to engage with a ring gear 246 disposed about the keyed drive stage 520, as described further herein.

On the second, torque output end of the keyed drive stage 520, an opening 252 having a keyed internal surface 254 is provided, as described elsewhere herein. The opening 252 may be configured to matingly receive and engage a keyed male feature such as, e.g., an external keyed surface 140 on the end 134 of a lead screw 122, to transmit torque. The keyed opening 252 may have any cross-sectional geometry configured to transmit torque, for example, square, rectangle, hexagon, or star. The keyed drive stage 520 may further include a retainer 150 as described herein relative to, e.g., FIG. 5.

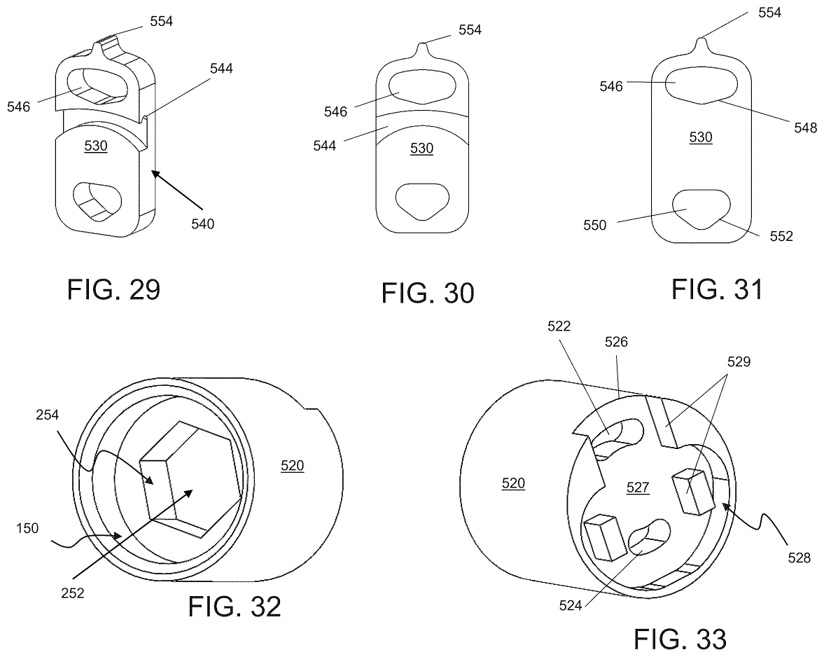

A keeper 530, shown in detail in FIGS. 29-31, and in context in FIGS. 26, 27, and 34-36, may be configured to be disposed within the recess 527 in the keyed drive stage 520. The keeper 530 is configured to move from a locked position to an unlocked position in response to rotation of the driver, e.g., magnet housing 204. In particular, the keeper 330 is configured to rotate and to translate in response to the rotation of the driver, thereby moving from the locked position to the unlocked position. In the locked position, the keeper 530 is configured to resist, reduce, minimize, or in certain embodiments prevent rotation of the keyed drive stage 520 and the magnet housing 204 under a load on the lead screw 122. In the unlocked position, the keeper 530 is configured to permit rotation of the keyed drive stage 520 and the driver, e.g., magnet housing 204.

As best seen in FIGS. 29-31, the keeper 530 includes a body configured to be received within the recess 527. The keeper 530 may include drive surfaces 540 which are configured to cooperate with the vertical guide surfaces 529 on the recess 527 to allow the keeper 530 to translate along the recess 527, and to allow the keeper 530 to drive rotation of the keyed drive stage 520. The drive surfaces 540 may be straight or substantially straight surfaces that are parallel or substantially parallel to one another, and perpendicular to the axis of rotation of the keyed drive stage 520.

The keeper 530 may include at least one lock tooth 554 disposed on a first end of the keeper 530, and extending axially therefrom. The lock tooth 554 is configured to releasably mesh with or engage the internal teeth 248 on the inner wall 244 of a ring gear 248 in use (see FIGS. 35-36). The lock tooth maybe any mating male/female shapes for example square or triangular tooth shapes. The keeper 530 further includes a first detent 546, which may be disposed at the first end, near the lock tooth 554, and may be laterally aligned with the lock tooth 554. The first detent 546 may be configured to engage the first drive pin 502 in use. The keeper 530 may further include a second detent 550, disposed at the second end of the keeper 530 opposite the first end, the second detent 550 being configured to engage the second drive pin 504. The second detent may also be laterally aligned with the first detent 546 and the lock tooth 554. It is noted that the first detent 546 may alternatively engage the second drive pin 504, and the second detent 550 may engage the first drive pin to equal effect.

Each of the first detent 546 and the second detent 550 may include a respective ramp surface 548, 552, along which the respective drive pin 502, 504 is configured to travel. The first ramp 548 and second ramp 552 may each be configured to be curved or angled, similar to first ramp 348 and second ramp 352 (see FIG. 17), having an approximately or substantially u-shaped or v-shaped ramp surface. Each ramp 548, 552 may include a zenith which is laterally aligned with the other, and also laterally aligned with the lock tooth 554 on the keeper 530. Accordingly, the drive pins 502, 504 disposed in each of the first detent 546 and the second detent 550 are disposed in the zeniths of the first ramp 548 and the second ramp 552 and in line with the lock tooth 554 when the keeper 530 is in the locked position. The drive pins 502, 504 disposed in each of the respective first and second detents 546, 550 are configured to move up the respective first ramp 548 and the second ramp 552 when the keeper 530 moves into the unlocked position. In certain embodiments, the first detent 546 and the second detent 550 each extend through a full thickness of the keeper 530.

As shown in FIGS. 29-30, the keeper 530 further includes a spring guide slot 544. Unlike the detents 546 and 550, the spring guide slot 544 may extend through only a partial thickness of the keeper 530. The spring guide slot 344 may be curved, and may be configured to receive a portion of a biaser or spring 506 (see FIG. 27) therein. As such, the shape and size of spring guide slot 544 may be complementary to that of a portion of spring 506.

The lock mechanism 500 further includes the biaser or spring 506, which may be disposed partially within the spring guide slot 344, and partially within a spring contour 528 in the recess 527. The biaser 506 may be configured to bias the keeper 530 away from the spring contour 528. The recess 527 may be open at the end 526 that is opposite the spring contour 528. Thus, the biaser may be configured to bias the keeper 530 across, or in a direction substantially perpendicular to the rotational axis of the magnet assembly, such that the lock tooth 554 extends beyond the outer circumference of the keyed drive stage 520. In use, this allows the lock mechanism 500 to achieve the locked position shown in FIG. 35.

With reference to FIGS. 26-27 and 34, the lock mechanism 500 may further include a radial bearing 508, which may be disposed about at least a portion of the magnet housing 204, and may be configured to assist in maintaining the spatial relationships between the magnet housing 204, the keeper 530, and the keyed drive stage 520.

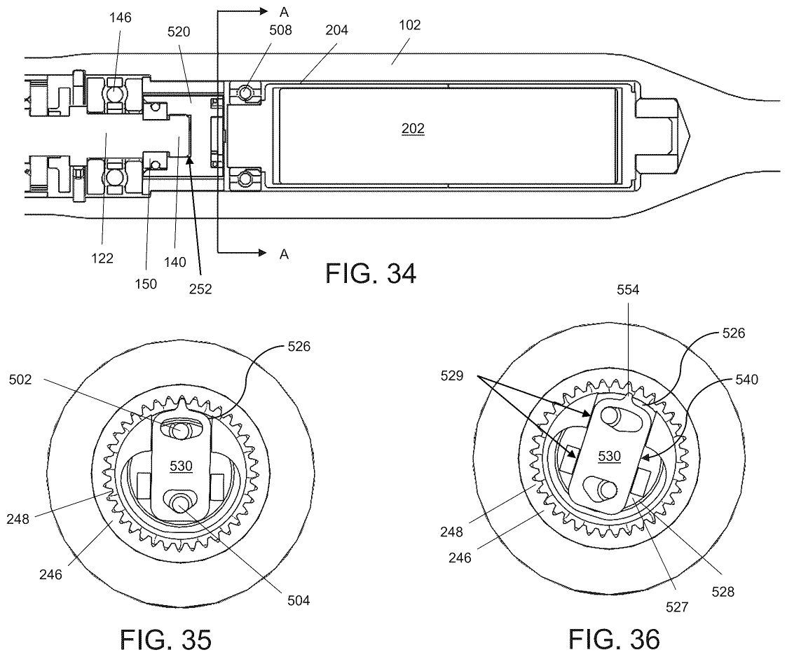

In operation, the lock mechanism 500 including the keeper 530, defaults to a locked position when the driver, e.g., magnet housing 204, is stationary (see FIG. 35). In the locked position, the biaser 506 is configured to bias the keeper 530 toward the open end 526 and against the spring contour 528 of the recess 527. Under the force of biaser 506 in its expanded condition, the keeper 530 translates across the rotational axis toward and at least partially through the open end 526 of the recess 527, such that the lock tooth 554 on the keeper 530 extends beyond an outer circumference of the keyed drive stage 520 and is biased into meshed engagement with the teeth 248 on the inner surface 244 of the ring gear 246. Due to the geometry of the first and second detents 546, 550, the first and second drive pins 502, 504 are disposed in the zenith positions of each of the respective ramps 348, 352. In this position, the keeper 530 resists rotation of the keyed drive stage 520 and under a load on the lead screw 122 (see FIG. 34).

Upon, and in response to rotation of the driver, e.g., the magnet housing 204, the keeper 530 transitions from the locked position to the unlocked position. As the magnet housing 204 rotates, either in a clockwise or counterclockwise direction, the drive pins 502, 504 move out of the zenith position following the curve or slope of ramps 548, 552. In the embodiment shown in FIG. 36, the magnet housing 204 has rotated in a counter clockwise direction, although the keeper 530 would function analogously if the rotation were clockwise. This rotation causes the keeper 530 to translate axially relative to the recess 527 in the direction away from the open end 526. Relative to this translation motion, the lock tooth 554 is disposed on a trailing end of the keeper 530. This translation causes the lock tooth 554 to disengage from the ring gear 246, and the biaser 506 to be compressed by the keeper 530, and in particular by spring guide slot 544. With the lock tooth 554 now disengaged from the teeth 248 of the ring gear 246, the keeper 530 moves to its unlocked position, in which the keyed drive stage 520, and therefore the lead screw 122, are free to rotate relative to the ring gear 246.

Upon, and in response to a cessation of rotation of the magnet housing 204, the opposite process occurs. In the absence of rotation of the magnet housing 204, and under the biasing force of biaser 506, the drive pins 502, 504 move downward along the ramps 548, 552 to return to the zenith position depicted in FIG. 35, and the keeper 530 translates axially relative to the recess 527. The lock tooth 554 is on the leading end of the keeper 530 with respect to this translation motion. Lock tooth 554 engages the teeth 248 of the ring gear 246, thereby preventing rotation of the keyed drive stage 520. Where the keyed drive stage 520 is rotationally fixed, so too is the lead screw 122. In this manner, regardless or independently of the load placed on the lead screw 122, back-driving and resulting distraction or compression loss are resisted, and may in some embodiments be prevented.

As used herein, the terms “first,” “second,” and the like, do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by the context (e.g., includes the degree of error associated with measurement of the particular quantity). The suffix “(s)” as used herein is intended to include both the singular and the plural of the term that it modifies, thereby including one or more of that term (e.g., the metal(s) includes one or more metals). Ranges disclosed herein are inclusive and independently combinable (e.g., ranges of “up to about 25 mm, or, more specifically, about 5 mm to about 20 mm,” is inclusive of the endpoints and all intermediate values of the ranges of “about 5 mm to about 25 mm,” etc.).

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An implantable medical device comprising:
a driver;
a driven gear system; and
a keeper, the keeper comprising:
a body;
a keyed opening in the body, the keyed opening being configured to receive a complementarily keyed portion of a drive gear of the driven gear system, wherein the keeper and the drive gear are fixed to one another;
an opening in the body configured to receive a center pin of the drive gear of the driven gear system; and
wherein the keeper is configured to move from a locked position to an unlocked position in response to an actuation of the driver,
wherein, in the locked position, the keeper is configured to resist rotation of the drive gear and the driver under a load on the driven gear system, and in the unlocked position, permits rotation of the drive gear and the driver.

2. The implantable medical device of claim 1, wherein the keeper is further comprising:
a lock tooth disposed on a first end of the body, the lock tooth being configured to releasably mesh with teeth on a ring gear of the driven gear system, and
rotate to move from the locked position to the unlocked position;
translate axially relative to the center pin in a direction opposite the first end, in response to the rotating of the keeper; and
disengage the lock tooth from the ring gear of the driven gear system in response to the translating,
thereby moving the keeper to the unlocked position.

3. The implantable medical device of claim 2, wherein the keeper further comprises a first face configured to engage the driver, and a second face configured to engage the driven gear system.

4. The implantable medical device of claim 3, wherein the opening extends from the first face through the body to the second face, and wherein the keyed opening extends from the second face through a partial thickness of the keeper.

5. The implantable medical device of claim 4, wherein the opening and the keyed opening are fluidly coupled,
wherein the keyed opening is bounded by drive surfaces configured to drive rotation of the keyed portion of the drive gear, and the opening is bounded by shaft relief surfaces configured to permit translation of the center pin, and
wherein the keeper further comprises a stepped surface between the drive surfaces and the shaft relief surfaces.

6. The implantable medical device of claim 3, wherein the keeper further comprises:
a first detent disposed on the first face, the first detent being disposed between the opening and the first end of the body, the first detent comprising a first ramp surface, and
a second detent disposed on the first face at a second end thereof, opposite the first end, the second detent comprising a second ramp surface,
wherein each of the first detent and the second detent is configured to receive a drive pin disposed on an end of the driver.

7. The implantable medical device of claim 6, wherein the keeper further comprises:
a hole disposed in the second end of the body and open to the keyed opening, the hole being configured to receive a biaser,
wherein, in the locked position, the lock tooth is configured to engage the ring gear, and to maintain such engagement under biasing force from the biaser in an expanded condition, and
wherein, in the unlocked position, the lock tooth is configured to disengage from the ring gear, in response to a compression of the biaser upon rotation of the driver and the keeper.

8. The implantable medical device of claim 7, wherein the first ramp and the second ramp are each substantially v-shaped, and a zenith of the first ramp, a zenith of the second ramp, and the lock tooth are laterally aligned,
such that the drive pins disposed in each of the first detent and the second detent are disposed in the zeniths of the first ramp and the second ramp when the keeper is in the locked position, and
such that the drive pins disposed in each of the first detent and the second detent move up the respective first ramp and the second ramp when the keeper moves into the unlocked position.

9. The implantable medical device of claim 8, wherein the first detent and the second detent are further configured to permit rotation of the driver relative to the keeper to an extent limited by a length of the first ramp and the second ramp.

10. The implantable medical device of claim 6, wherein the first detent and the second detent each extend through a partial thickness of the keeper.

11. A lock mechanism configured for locking and unlocking rotation of a driver and a driven gear system in an implantable distraction and compression system, the lock mechanism comprising:
a driver comprising a first drive pin and a second drive pin each extending axially from a first end of the driver;
a drive gear configured to be driven by the driver, the drive gear comprising a keyed portion, and
a keeper disposed over the drive gear and configured to move between a locked position and an unlocked position, the keeper comprising:
a keyed opening configured to rotatably engage the keyed portion of the keyed drive shaft,
a first detent and a second detent configured to engage the first drive pin and the second drive pin, and
a biaser configured to bias the keeper across a longitudinal axis of the driver and the drive gear,
wherein, in the locked position, the keeper resists rotation of the drive gear and the driver under a load on the driven gear system, and in the unlocked position, permits rotation of the drive gear and the driver.

12. The lock mechanism of claim 11, wherein, in the locked position, the biaser is configured to bias the keeper and a lock tooth disposed on a first end of the keeper, the lock tooth being configured to releasably engage a ring gear of the driven gear system, the lock tooth configured to be in a meshed engagement with the ring gear, and in the unlocked position, the biaser is configured to be compressed by the keeper upon rotation of the driver, thereby releasing engagement between the lock tooth and the ring gear and permitting the driver and the drive shaft to rotate.

13. The lock mechanism of claim 11, wherein the driver further comprises a central recess open to the first end of the driver and disposed between the first drive pin and the second drive pin, and
wherein the drive gear further comprises a central pin and a gear, each coupled to the keyed portion of the drive gear at opposing ends thereof, wherein the central pin is disposed at least partly within the central recess of the driver.

14. The lock mechanism of claim 11, further comprising a hole extending from the keyed opening through the second end of the keeper, and into the keyed portion,
wherein the second end is opposite the first end, and
wherein the biaser is disposed within the hole.

15. The lock mechanism of claim 11, further comprising a radial bearing configured to maintain a coaxial relationship between two or more of the driver, the keeper, and the keyed drive gear.

16. The lock mechanism of claim 11, wherein each of the first and the second detents comprise a ramp surface along which the respective drive pin is configured to travel.

17. The lock mechanism of claim 11, wherein the driver comprises a cylindrical permanent magnet configured to be rotated by the application of a magnetic field, and a magnet housing disposed about the cylindrical permanent magnet.

18. The lock mechanism of claim 11, wherein the gear is a sun gear, and the driven gear system is a planetary gear system, and
wherein the sun gear is configured to engage the ring gear via a plurality of planetary gears.

19. The lock mechanism of claim 11, wherein the keyed portion has a square cross-sectional shape, and the keyed opening has a corresponding and complementary square cross-sectional shape.

20. An implantable distraction and compression system comprising:
a housing configured to be attached to a first bone portion, the housing having a driver, a driven gear system, and a lead screw positioned therein, wherein the lead screw is coupled to the driver via the driven gear system such actuation of the driver causes movement of the lead screw;
a rod configured to be attached to a second bone portion and configured to interact with the lead screw such that, upon movement of the lead screw, the rod distracts or contracts relative to the housing; and
a lock mechanism configured to lock and unlock movement of the driver and the driven gear system, the lock mechanism comprising:
a drive gear configured to be driven by the driver, and to input torque into the driven gear system, the drive gear comprising a keyed portion, and
a keeper engaged with the driver and with the keyed portion to move between a locked position and an unlocked position in response to actuation of the driver,
wherein, in the locked position, the keeper resists rotation of the drive gear and the driver under a load on the lead screw, and in the unlocked position, permits movement of the drive gear and the driver.
